(19) United States
(12) Patent Application Publication
(10) Pub. No.: US 2023/0033638 A1
(43) Pub. Date: Feb. 2, 2023

(54) SOLUBLE REAR LAYER FOR OTF

(71) Applicant: LTS LOHMANN THERAPIE-SYSTEME AG, Troisdorf (DE)

(72) Inventor: Markus MÜLLER, Troisdorf (DE)

(21) Appl. No.: 17/787,874

(22) PCT Filed: Dec. 18, 2020
(86) PCT No.: PCT/EP2020/087195

§ 371 Date: Jun. 21, 2022

(30) Foreign Application Priority Data
Dec. 20, 2019 (DE) 10 2019 135 432.3

(51) Int. Cl. A61K 9/70; A61K 31/165; A61K 31/135; A61K 47/14; A61K 47/26; A61K 9/00

(52) U.S. Cl. CPC A61K 9/7007; A61K 9/006; A61K 31/135; A61K 31/165; A61K 47/14; A61K 47/26

(57) ABSTRACT

The invention relates to a multi-layer oral thin film comprising a matrix layer, which contains at least one polymer and at least one pharmaceutically active agent, and at least one backing layer, wherein the at least one backing layer comprises at least one polymer containing free carboxyl groups, wherein 10 to 100% of the free carboxyl groups of the at least one polymer comprising free carboxyl groups are present in a neutralised form as a salt; to a method for production thereof, and to use thereof as a medicament.

The present invention relates to a multi-layer oral thin film, to a method for production thereof, and to the use thereof as a medicament.

Oral thin films are thin films containing at least one pharmaceutically active agent that are placed directly in the oral cavity or against the oral mucosa and dissolve or macerate there and in so doing deliver the active agent. These films are, especially, thin, one- or multi-layer, active agent-containing polymer-based films which, when applied to a mucous membrane, especially the oral mucosa, can deliver the active agent directly into same. The very good blood supply to the oral mucosa ensures a rapid transfer of the active agent into the bloodstream. This dosage system has the advantage that the active agent is resorbed for the most part by the mucous membrane, thus avoiding the first-pass effect, which occurs in the case of the conventional dosage form of an active agent in tablet form. The active agent may be dissolved, emulsified or dispersed in the film.

Oral thin films known from the prior art have the disadvantage that if they are intended to remain for a longer time at a point on the mucous membrane of a patient, they are exposed to a permanent erosion. This leads to a large part of the material being swallowed and thus not remaining at the application site for the desired duration. However, the residence time may well be of decisive importance for the transmucosal transport of the pharmaceutically active agent.

A protective layer on the rear side can prevent liquid from penetrating the formulation and dissolving it too quickly, so that the active agent remains at the application site for the maximum time to achieve the greatest possible permeation through the mucosa or a delayed release. Another effect of the backing layer is that it prevents the administered film from detaching from the application site and adhering elsewhere, such as the teeth.

Insoluble or very slowly soluble polymers or polymer films are often used as the material for such backing layers. However, these have the disadvantage that they had to be removed or swallowed once the application was complete.

Backing layers made of slowly soluble polymers also have the disadvantage that they are based on long-chain, high-molecular polymers. These are difficult to process due to their high viscosity (long drying times, irregular films). Furthermore, they tend to increase the viscosity of the saliva in the oral cavity, resulting in a slimy feeling in the oral cavity.

The aim of the present invention lies in overcoming the above-mentioned disadvantages of the prior art. Especially, it is the aim of the present invention to provide a multi-layer oral thin film having at least one backing layer, the dissolution time of which can be varied and thus adjusted, and which can be dissolved without residue and without producing an unpleasant mouthfeel.

The above aim is addressed by a multi-layer oral thin film according to claim 1, comprising a matrix layer, which contains at least one polymer and at least one pharmaceutically active agent, and at least one backing layer, wherein the at least one backing layer comprises at least one polymer containing free carboxyl groups, wherein 10 to 100% of the free carboxyl groups of the at least one polymer comprising free carboxyl groups are present in a neutralised form as a salt.

Such a multi-layer oral thin film has the advantage that, by selectively adjusting the degree of neutralisation of the at least one polymer comprising free carboxyl groups, a backing layer can be provided which has dissolved without residue after a desired time. Since the polymer is preferably water-insoluble in the non-neutralised state and is highly water-soluble in the fully neutralised state, the dissolution rate can be adjusted as desired from insoluble to rapidly soluble. The backing layer thus protects the applied film reliably and for the desired duration by way of slow solubility.

Even brushing over with the tongue cannot result in increased erosion, as dissolution cannot be accelerated mechanically, but depends on the buffering capacity of the existing saliva and the subsequently produced saliva.

Furthermore, it is advantageous that, due to the good solubility in the neutralised state, the backing layer does not trigger an unpleasant mouthfeel.

In addition, the backing layer can be coloured with colouring agent to achieve better visibility.

Furthermore, it is possible to incorporate flavourings to improve the mouthfeel during application, which is a major advantage over other films.

In the present description of the oral thin film, the word “comprising” can also mean “consisting of”.

The term “backing layer” is understood to mean a layer of the multi-layer oral thin film that is one of the outermost layers of the multi-layer oral thin film.

The at least one polymer comprising free carboxyl groups is preferably a polymer which is difficult to dissolve, very difficult to dissolve or practically insoluble in water according to the following table. Solubility at 15° C. to 25° C.:

| Description | V(solvent) in ml per g of substance | g·l⁻¹ solvent |
|---|---|---|
| very easily soluble | < 1 | > 1000 |
| easily soluble | 1 to 10 | 100 to 1000 |
| soluble | 10 to 30 | 33 to 100 |
| slightly soluble | 30 to 100 | 10 to 33 |
| difficult to dissolve | 100 to 1000 | 1 to 10 |
| very difficult to dissolve | 1000 to 10000 | 0.1 to 1 |
| practically (almost) insoluble | > 10000 | < 0.1 |

It has been shown that the solubility in water of polymers comprising free carboxyl groups can be varied by partial neutralisation or the addition of at least one base. Applied to the backing layer of the multi-layer oral thin film, this means that the disintegration time of the backing layer in the patient’s mouth can be varied.

The multi-layer oral thin film according to the invention is preferably characterised in that at least 10%, especially preferably from 15 to 100% of the free carboxyl groups of the at least one polymer comprising free carboxyl groups are present in neutralised form as a salt.

Preferably, 15 to 100% or 20 to 100% or 25 to 100% or 30 to 100% or 35 to 100% or 40 to 100% or 45 to 100% or 50 to 100% or 60 to 100% or 70 to 100% or 80 to 100% or 90 to 100% of the free carboxyl groups of the at least one polymer comprising free carboxyl groups are present in neutralised form as a salt.

Preferably, 10 to 100% or 15 to 100% or 20 to 95% or 25 to 90% or 30 to 85% or 35 to 80% or 40 to 75% or 45 to 70% or 50 to 65% or 55 to 60% of the free carboxyl groups of the at least one polymer comprising free carboxyl groups are present in neutralised form as a salt.

If 0% of the free carboxyl groups of the at least one polymer comprising free carboxyl groups are in neutralised form, the polymer is preferably insoluble. If about 10% of the free carboxyl groups of the at least one polymer comprising free carboxyl groups are neutralised, the polymer preferably has a pH of about 4 and is thus very slowly soluble.

If about 15% to 100% of the free carboxyl groups of the at least one polymer comprising free carboxyl groups are in neutralised form, the polymer preferably has a pH of about 4.6 to 7.

The multi-layer oral thin film according to the invention is further preferably characterised in that the at least one backing layer has a pH of from 3.5 to 7.5, preferably from 4 to 7, especially from 4.5 to 6.5.

The pH of the backing layer corresponds to the pH of the solution or suspension of an aqueous solution of all the ingredients of the backing layer at 20° C. The aqueous solution can comprise exclusively water as a solvent here, but also mixtures of water with organic solvents. In principle, all water-miscible organic solvents are suitable as organic solvents. Preferred are ketones, alcohols and/or esters, especially acetone or ethanol.

If more than one polymer comprising free carboxyl groups is present in the backing layer, the above values for the degree of neutralisation of the free carboxyl groups refer to the sum of all carboxyl groups present.

The multi-layer oral thin film according to the invention is also preferably characterised in that the at least one polymer comprising free carboxyl groups is provided in the backing layer in an amount of 10 to 99 wt.%, preferably of 20 to 95 wt.%, especially preferably of 25 to 90 wt.%, in relation to the total weight of the backing layer.

Mixtures of several polymers comprising free carboxyl groups can also be used. In this case, the sum of all polymers comprising free carboxyl groups is 10 to 99 wt.%, preferably 20 to 95 wt.%, especially preferably 25 to 90 wt.%, in relation to the total weight of the backing layer.

The multi-layer oral thin film according to the invention is further preferably characterised in that the content of free carboxyl groups in the polymer comprising free carboxyl groups is 10 to 40 wt.%, preferably 15 to 35 wt.%, especially preferably 20 to 30 wt.%, in relation to the mean polymer mass.

If more than one polymer comprising free carboxyl groups is present in the back layer, these mentioned values relate to the sum of all carboxyl groups present.

The at least one polymer comprising free carboxyl groups preferably comprises a polymer based on (meth)acrylic acid and/or based on a copolymer of (meth)acrylic acid and (meth)acrylates.

Very especially preferably, the at least one polymer comprising free carboxyl groups comprises a copolymer of (meth)acrylic acid and (meth)acrylates, very especially preferably a (meth)acrylic acid/ethyl acrylate copolymer.

Suitable polymers are available, for example, under the trade names Kollicoat L100, Kollicoat L100-55, Kollicoat MAE Kollicoat Smart Seal (from BASF) or Eudragit L, Eudragit S (Evonic) or Acryl-EZE (Colorcon).

These polymers have the advantage that they are almost insoluble in water and their solubility can be easily varied by adding at least one base. Some of these polymers are already pre-neutralised by the manufacturer.

The multi-layer oral thin film according to the invention is further preferably characterised in that the free carboxyl groups of the at least one polymer comprising free carboxyl groups have been neutralised by addition of at least one base.

The type of base is not limited, but in principle any pharmaceutically acceptable base can be used. Especially preferably, the base comprises a hydroxide, especially an alkali metal hydroxide or alkaline earth metal hydroxide. The use of NaOH is very especially preferred.

Preferably, the added amount of the at least one base is such that the equivalence ratio of the base to the free carboxyl groups of the at least one polymer comprising free carboxyl groups is 1:10 to 2:1, preferably 1:10 to 1:1.

If NaOH is added as a base, the amount added may preferably be such that 0.5 to 15 wt. % of NaOH, in relation to the weight of the at least one polymer comprising free carboxyl groups, is added.

The multi-layer oral thin film according to the invention is further preferably characterised in that the backing layer comprises at least one plasticiser.

Suitable plasticisers here comprise citric acid ester, alpha tocopherol, benzyl benzoate, butyl stearate, chlorobutanol, dibutyl phalate, dibutyl sebacate, diethyl phalate, dimethyl phthalate, dipropylene glycol, glycerol, glycerol monostearate, polyethylene glycol, propylene glycol, stearic acid, triacetin and/or tricapryline, especially preferably triethyl citrate.

The multi-layer oral thin film according to the invention is preferably characterised in that the at least one plasticiser is provided in the backing layer of the multi-layer oral thin film in an amount of 1 to 20 wt.%, preferably of 5 to 15 wt.%, especially preferably of 8 to 12 wt.%, in relation to the total weight of the backing layer.

The multi-layer oral thin film according to the invention is further preferably characterised in that the matrix layer comprises at least one water-soluble polymer.

Water-soluble polymers comprise chemically very different natural or synthetic polymers, the common feature of which is their solubility in water or aqueous media. A precondition is that these polymers have a number of hydrophilic groups sufficient for the water solubility and are not crosslinked. The hydrophilic groups may be non-ionic, anionic, cationic and/or zwitterionic.

Water-soluble polymers preferably have a solubility in water according to the above table of at least “soluble”.

The at least one water-soluble polymer is preferably selected from the group consisting of starch and starch derivatives, dextrans, cellulose derivatives, such as carboxymethyl cellulose, hydroxypropyl cellulose, hydroxyethyl cellulose, hydroxypropyl methylcellulose, hydroxypropyl ethyl cellulose, sodium carboxymethyl cellulose, ethyl or propyl cellulose, polyacrylic acids, polyacrylates, polyvinylpyrrolidones, vinylpyrrolidone/vinyl acetate copolymer, polyvinyl alcohols, polyethylene oxide polymers, polyacrylamides, polyethylene glycols, gelatines, collagen, alginates, pectin, pullulan, tragacanth, chitosan, alginic acid, arabinogalactan, galactomannan, agar, agarose, carrageenan, and natural gums.

Especially preferably, the at least one water-soluble polymer comprises vinvlpyrrolidone/vinyl acetate copolymer.

The multi-layer oral thin film according to the invention is preferably characterised in that the at least one polymer, preferably the water-soluble polymer, is provided in the matrix layer of the multi-layer oral thin film in an amount of 5 to 95 wt.%, preferably of 10 to 80 wt.%, in relation to the total weight of the matrix layer.

Embodiments of the multi-layer oral thin film according to the invention, wherein the at least one polymer, preferably the water-soluble polymer, is provided in the matrix layer of the multi-layer oral thin film in an amount of 5 to 50 wt.%, preferably of 10 to 40 wt.%, in relation to the total weight of the matrix layer, are also possible.

The at least one pharmaceutically active agent is not subject in principle to any limitation, but is preferably selected from all pharmaceutically active agents that are suitable for oral and/or transmucosal application.

According to the present invention, all pharmaceutically acceptable salts and solvates of the particular pharmaceutically active agent are also subsumed under the pharmaceutically active agent.

Active agents are preferably selected from the group consisting of the active agent classes of analgesics, hormones, hypnotics, sedatives, antiepileptics, analeptics, psychoneurotropic drugs, neuro-muscle blockers, antspasmodics, antihistamines, antiallergics, cardiotonics, antiarrhythmics, diuretics, hypotensives, vasopressors, antidepressants, antitussives, expectorants, thyroid hormones, sexual hormones, antidiabetics, antitumour active agents, antibiotics, chemotherapeutics and narcotics, however, this group is not conclusive.

The at least one pharmaceutically active agent is especially preferably ketamine and/or a pharmaceutically active salt or solvate thereof, preferably ketamine HCI.

In the present case, ketamine is understood to mean (S)-(±)-2-(2-chlorophenyl)-2-(methylamino)cyclohexan-1-one, (R)-(±)-2-(2-chlorophenyl)-2-(methylamino)cyclohexan-1-one, and the racemate (RS)-(±)-2-(2-chlorophenyl)-2-(methylamino)cyclohexan-1-one.

However, (S) ketamine or a pharmaceutically acceptable salt thereof, especially (S) ketamine HCl, is especially preferably present as a single stereoisomer of ketamine, since the analgesic and anaesthetic potency of (S) ketamine is approximately three times higher than that of the (R) form.

The multi-layer oral thin film according to the invention is also preferably characterised in that the at least one pharmaceutically active agent, preferably the ketamine, is provided in the matrix layer in an amount of 1 to 50 wt.%, preferably of 10 to 40 wt.%, especially preferably of 25 to 35 wt.%, in relation to the total weight of the matrix layer.

The multi-layer oral thin film according to the invention is also preferably characterised in that the matrix layer also comprises in each case at least one auxiliary substance selected from the group comprising colouring agents, flavourings, sweeteners, plasticisers, taste-masking agents, emulsifiers, enhancers, pH regulators, humectants, preservatives and/or antioxidants.

Each of these auxiliary substances is preferably contained in this layer in each case in an amount of about 0.1 to 40 wt.%, preferably of 0.1 to 30 wt.%, especially preferably of 0.1 to 15 wt.%, in relation to the total weight of the matrix layer.

The multi-layer oral thin film according to the invention is also preferably characterised in that the backing layer also comprises at least one auxiliary substance selected from the group comprising colouring agents, flavourings, sweeteners, taste-masking agents, emulsifiers, enhancers, pH regulators, humectants, preservatives and/or antioxidants.

Each of these auxiliary substances is preferably contained in this layer in each case in an amount of about 0.1 to 40 wt.%, preferably of 0.1 to 30 wt.%, especially preferably of 0.1 to 15 wt.%, in relation to the total weight of the backing layer.

The oral thin film according to the invention preferably has an area of from about 0.5 cm² to about 10 cm², more preferably from about 2 cm² to about 8 cm².

The oral thin film according to the invention is preferably characterised in that the mass per unit area of the oral thin film is approximately 10 to 500 g/m², preferably approximately 100 to 400 g/m².

The area density of the backing layer is important for controlling the dissolution behaviour and the function of the backing layer to protect the active agent from dissolving in saliva. A certain thickness is required to ensure sufficient protection of the active agent as well as a sufficient dissolution time, which should normally be at least as long as the active permeation requirement and thus the dissolution time of the matrix layer.

Furthermore, it is also difficult to coat, especially, very thin layers with sufficient accuracy. On the other hand, thick layers may not only cause an unpleasant sensation in the oral cavity, but may also be difficult to produce and may cause the layer to take too long to dissolve for the desired (e.g. overnight) application. On the whole, it is preferred that the backing layer has an area density of at least 10 g/m², more preferably at least 20 g/m² or most preferably at least 30 g/m², or an area density of less than or equal to 400 g/m², more preferably less than or equal to 350 g/m² or most preferably less than or equal to 300 g/m², or an area density of from 10 to 400 g/m², more preferably from 20 to 350 g/m² or most preferably from 30 to 300 g/m².

This preferably corresponds to a layer thickness of preferably from about 10 µm to about 500 µm, especially preferably from about 20 µm to about 300 µm.

The oral thin film according to the invention is preferably characterised in that in certain embodiments the carrier layer dissolves in 5 minutes or more, preferably in 10 minutes or more, and more preferably in 15 minutes or more, or in 12 hours or less, preferably in 8 hours or less, and more preferably in 4 hours or less, or in 5 minutes or 12 hours, preferably in 10 minutes or 8 hours, and more preferably in 15 minutes or 4 hours after administration of the oral thin film to a patient.

It is also preferred that the backing layer is formed in water, in artificial or natural saliva or in any other aqueous medium, preferably in a buffered aqueous medium, at 37° C. and 150 rpm, in 5 minutes or more, preferably in 10 minutes or more, preferably in 15 minutes or more, and more preferably in 30 minutes or more, and in 15 hours or less, preferably in 12 hours or less, and more preferably in 10 hours or less, or in 10 minutes or 15 hours, preferably in 15 minutes or 12 hours, and more preferably in 30 minutes or 10 hours.

Especially, the backing layer may be equal to or larger than the matrix layer in terms of size. Thus, in certain embodiments the size of the backing layer and the size of the matrix layer are the same, while in other embodiments the backing layer is larger than the surface area of the matrix layer and increases in size. While a layered structure with the same size of backing layer and matrix layer is easier to produce, since a two-layer sheet can be punched out to provide the layered structure, a layered structure with a backing layer larger than the matrix layer is more difficult to produce, but also offers the advantage that there is less risk of active leakage, since the edge of the matrix layer is also covered by the carrier layer.

The present invention also relates to a method for producing the oral thin film according to the invention. The method comprises the steps of:
a) providing at least one active-agent-containing matrix layer, comprising the steps of
   a1) producing a suspension or suspension comprising the at least one polymer and the at least one pharmaceutically active agent, and
   a2) spreading out and drying the solution or suspension obtained in accordance with step a1);
b) providing at least one backing layer, comprising the steps of
   b1) producing a solution or suspension comprising at least one polymer comprising free carboxyl groups, wherein 10 to 100% of the free carboxyl groups of the at least one polymer comprising free carboxyl groups are present in a neutralised form as a salt;
   b2) spreading out and drying the solution obtained in accordance with step b1);
c) joining together the active-agent-containing matrix layer obtained in accordance with a) and the backing layer obtained in accordance with b) in order to obtain a multi-layer oral thin film.

The polymer used in step b) may be at least partially neutralised by the addition of at least one base as described above. Alternatively, a polymer that has already been pre-neutralised by the manufacturer may be used.

The two films can be joined by methods commonly known to a person skilled in the art. For example, a further film can be applied to a first film by means of coating, it being irrelevant which film is coated on which film, as long as the backing layer forms one of the outermost layers. Further, the two layers can be connected together by an adhesive layer. Furthermore, the two layers can be connected together by means of heat.

The present invention also relates to a multi-layer oral thin film obtainable by the method described above.

In addition, the present invention relates to a multi-layer oral thin film, as described above or obtainable by the above-described method, as a medicament.

The present invention additionally relates to a multi-layer oral thin film, as described above or obtainable by the above-described method, as a medicament for use in the treatment of pain and/or depressions, especially to reduce the risk of suicide and/or for use as a general anaesthetic, preferably to initiate and carry out general anaesthesia, or as a supplement in the case of local anaesthesia and/or as an analgesic.

The preferred embodiments described above for the multi-layer thin film according to the invention are also applicable for the method according to the invention, the multi-layer oral thin film obtained by this method, and use of said multi-layer oral thin film as a medicament.

### DESCRIPTION OF THE DRAWINGS

FIG. 1: Dissolution times of formulations without a backing layer,` with a PET backing layer, and with a backing layer according to the invention.

The invention will be described in greater detail hereinafter on the basis of non-limiting examples.

### EXAMPLES

### Example 1

A backing layer is produced as follows: Formulation of the composition:

Table 1

| Ingredient | Function | Proportion [%] |
|---|---|---|
| Kollicoat MAE 100-55 | Polymer | 42 |
| Kollicoat MAE 100 P | Polymer | 42 |
| Triethyl citrate | Plasticiser | 10 |
| Sucralose | Taste corrector | 3 |
| Saccharin Na | Taste corrector | 2 |
| Cherry Flavour | Taste corrector | 1 |

Solvent: ethanol:water 80:20
Target area density: approx. 50 g/m2

### Production Sequence

The solvents are put together and the plasticiser and flavouring are added. Sweeteners are then dissolved one after the other and the polymers are sprinkled in and dissolved. The mass is now left to rest until it is bubble-free.

The result is a homogeneous, slightly cloudy mass.

### Coating/Drying

The mass is applied to a siliconised liner and the resulting film is dried.

The result is a thin, stable, clear film.

### Further Processing

The resultant coating can now be further processed. For example, a further film, such as the matrix layer, can be applied to this backing layer by means of coating, bonding or heat.

### Example 2

In an initial investigation, three formulation variants were tested. Formulation 1 was the base without a backing layer. A formulation with PET backing (insoluble, 2) and the dissolving backing layer (3) according to the invention was then formed from the same mass.

Table 2

| Material | 1 | 2 | 3 |
|---|---|---|---|
| Matrix layer | | | |
| S ketamine HCI | 30.0 % | 30.0 % | 30.0 % |
| Kollidon VA 64¹ | 20.5 % | 20.5 % | 20.5 % |
| Kollicoat MAE² | 35.0 % | 35.0 % | 35.0 % |
| NaOH | 1.0 % | 1.0 % | 1.0 % |
| Saccharin Na | 2.0 % | 2.0 % | 2.0 % |
| Sucralose | 1.0 % | 1.0 % | 1.0 % |
| Cherry Flavour | 3.0 % | 3.0 % | 3.0 % |
| Glycerol | 7.5 % | 7.5 % | 7.5 % |
| Solvent | Purified water / ethanol | Purified water / ethanol | Purified water / ethanol |
| Backing layer | | | |
| PET backing layer | | FO PET 15 µm tsp. | |
| Kollicoat MAE backing layer | | | Kollicoat MAE backing layer: |
| Kollicoat MAE 100-55 | | | 42.0 % |
| Kollicoat MAE 100 P | | | 42.0 % |
| Triethyl citrate | | | 10.0 % |
| Cherry EU | | | 3.0 % |
| Saccharin Na | | | 2.0 % |
| Sucralose | | | 1.0 % |
| Solvent | | | Purified water / ethanol |

¹: Vinylpyrrolidone/vinyl acetate copolymer
²: (Meth)acrylic acid/ethyl acrylate copolymer

Table 3

| Formulation | Disintegration time [s] | Fully dissolved* [s] |
|---|---|---|
| 1 | 341 | 425 |
| 2 | 773 | 1122 |
| 3 | Not detectable | 742 |

* The full dissolution time refers to the dissolution of the matrix layer containing at least one pharmaceutically active agent.

The disintegration rate and the release of the active agent were examined.

The disintegration time was examined by means of a standard disintegration tester with sinker and is sufficiently known and described in the pharmacopoeias. The active agent was released by means of a standard dissolution tester using the “rotating cylinder” method. For this purpose, the OTFs were glued onto a cylinder that rotated in the release medium. The analysis was carried out via HPLC.

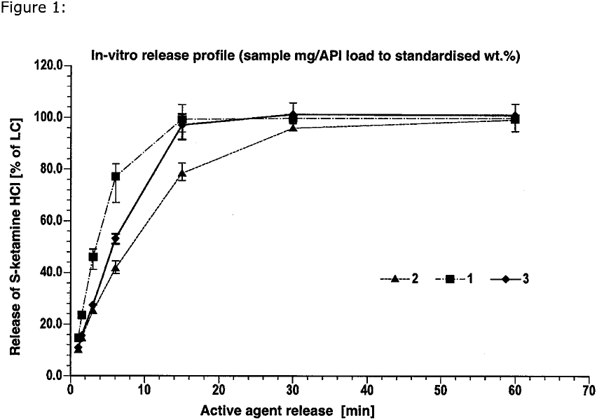

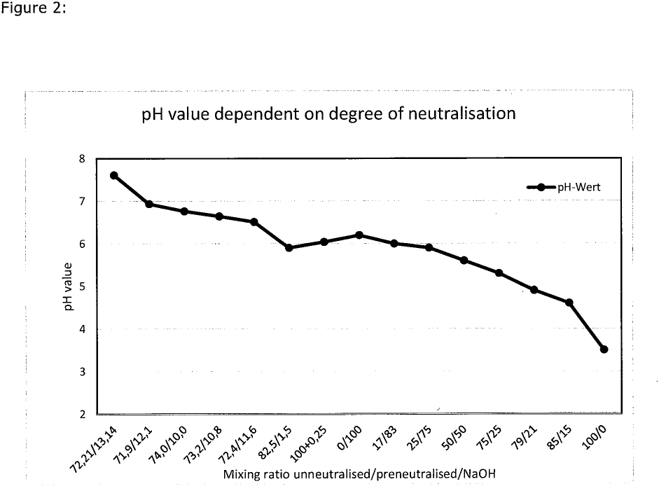

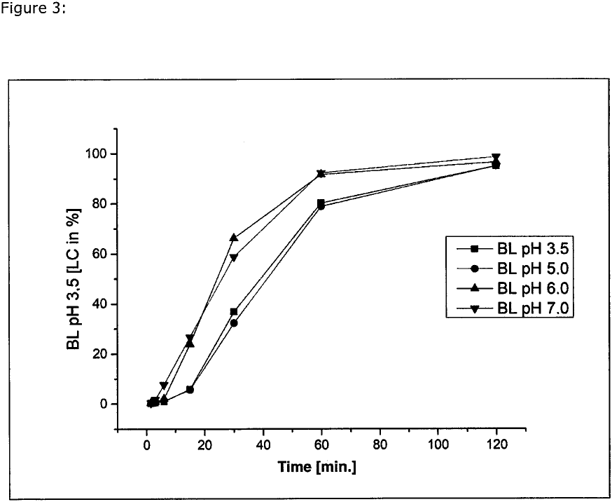

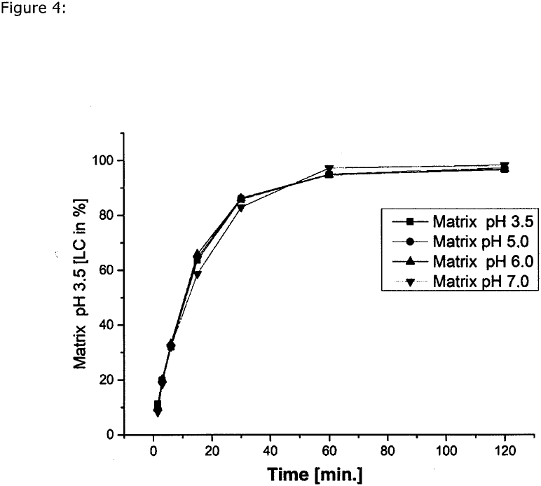

The tests confirmed that the backing layer of formulation 3 according to the invention dissolved and significantly slowed down the disintegration and the release of the active agent compared to the reference (1). The results are also shown in FIG. 1.

The polymer is neutralised by adding, dropwise, caustic soda (preferably as a solution in the appropriate solvent, which was also used for the polymer solution) to a polymer suspended in water or dissolved in a mixture of organic solvent and water (in this case Kollicoat L100-55). The increasing neutralisation reduces the solubility in the organic solvent and increases the solubility in water.

Here it is preferred that a certain amount of water is present, otherwise phase separation will occur and the polymer may precipitate as a solid component.

As the proportion of added caustic soda becomes very high at higher concentrations, a blend of pre-neutralised polymer (Kollicoat MAE 100P pre-neutralised by the manufacturer using NaOH) and non-neutralised polymer (Kollicoat MAE 100-55) was also used (see Table 4). The pH value of the polymer mixtures summarised in Table 4 is shown in FIG. 2.

Table 4

| Formulation | Kollicoat MAE 100-55 | Kollicoat MAE 100P | Ratio | NaOH [%] | pH value |
|---|---|---|---|---|---|
| 1 | | 72.21 | 72.21/13.14 | 13.14 | 7.61 |
| 2 | | 71.9 | 71.9//12.1 | 12.1 | 6.93 |
| 3 | | 74 | 74.0//10.0 | 10 | 6.76 |
| 4 | | 73.2 | 73.2//10.8 | 10.8 | 6.64 |
| 5 | | 72.4 | 72.4//11.6 | 11.6 | 6.51 |
| 6 | | 82.5 | 82.5//1.5 | 1.5 | 5.9 |
| 7 | | 100 | 100+/0.25 | 0.25 | 6.04 |
| 8 | 0 | 100 | 0/100 | 0 | 6.2 |
| 9 | 17 | 83 | 17/83 | 0 | 6 |
| 10 | 25 | 75 | 25/75 | 0 | 5.9 |
| 11 | 50 | 50 | 50/50 | 0 | 5.6 |
| 12 | 75 | 25 | 75/25 | 0 | 5.3 |
| 13 | 79 | 21 | 79/21 | 0 | 4.9 |
| 14 | 85 | 15 | 85/15 | 0 | 4.6 |
| 15 | 100 | 0 | 100/0 | 0 | 3.5 |

### Example 3

In a further test, the differences between different pH values were shown. For this purpose, a film of active agent with a similar composition as in previous tests was provided with backing layers with different degrees of neutralisation (pH values). The compositions (in wt.%) are summarised in Table 5.

Table 5

| Formulation | 1 | 2 | 3 | 4 |
|---|---|---|---|---|
| Matrix layer | | | | |
| S ketamine HCI | 30.0 % | 30.0 % | 30.0 % | 30.0 % |
| Kollidon VA 64 | 20.5 % | 20.5 % | 20.5 % | 20.5 % |
| Kollicoat MAE | 35.0 % | 35.0 % | 35.0 % | 35.0 % |
| NaOH | 1.0 % | 1.0 % | 1.0 % | 1.0 % |
| Saccharin Na | 2.0 % | 2.0 % | 2.0 % | 2.0 % |
| Sucralose | 1.0 % | 1.0 % | 1.0 % | 1.0 % |
| Cherry Flavour | 3.0 % | 3.0 % | 3.0 % | 3.0 % |
| Glycerol | 7.5 % | 7.5 % | 7.5 % | 7.5 % |
| Solvent | Purified water / ethanol | Purified water / ethanol | Purified water / ethanol | Purified water / ethanol |
| Kollicoat MAE backing layer | Kollicoat MAE pH 3.5: | Kollicoat MAE pH 5.0: | Kollicoat MAE pH 6.0: | Kollicoat MAE pH 7.0: |
| Kollicoat MAE 100-55 | 84.0 % | 66.4 % | 14.3 % | |
| Kollicoat MAE 100 P | | 17.6 % | 69.7% | 71.9 % |
| Triethyl citrate | 10.0 % | 10.0 % | 10.0 % | 10.0 % |
| Cherry EU | 3.0 % | 3.0 % | 3.0 % | 3.0 % |
| Saccharin Na | 2.0 % | 2.0 % | 2.0 % | 2.0 % |
| Sucralose | 1.0 % | 1.0 % | 1.0 % | 1.0 % |
| NaOH | | | | 12.1 % |
| Solvent | Purified water / ethanol | Purified water / ethanol | Purified water / ethanol | Purified water / ethanol |

The release was tested in two set-ups with the paddle over disk (TTS holder). In a first variation, the OTF was tested with a USP 5 TTS holder mesh size 40 (35 mm diameter) with the API side to a PET film and the backing layer to the release medium. The effect of the backing layer was able to be tested here. The measurement results are summarised in FIG. 3.

In a second variant, the set-up was basically the same, with the difference that the backing layer of the OTF was applied to the PET side to show the release profiles of the API matrix. The measurement results are summarised in FIG. 4.

### Example 5

Oral thin films were also prepared with agomelatine as the pharmaceutically active agent of the compositions shown in Tables 6 and 7.

Table 6

Agomelatine-containing matrix layer

| Ingredient | Ex. 3a | | Ex. 3b, 3c and 3d | | |
|---|---|---|---|---|---|
| | Amount [g] | Solid [%] | Amount [g] | | Solid [%] |
| Agomelatine | 0.50 | 9.96 | 3.50 | | 9.98 |
| Eucalyptol | 0.04 | 0.83 | 0.17 | | 0.49 |
| Menthol | 0.05 | 1.00 | 0.35 | | 1.00 |
| Methyl salicylate | 0.03 | 0.52 | 0.18 | | 0.51 |
| Novamint Fresh Peppermint | 0.18 | 3.52 | 1.24 | | 3.52 |
| Kolliphor RH 40 (Cremophor) | 0.12 | 2.43 | 0.72 | | 2.06 |
| FD&C Red #40 | 0.004 | 0.09 | 0.04 | | 0.10 |
| Sucralose | 0.03 | 0.50 | 0.18 | | 0.50 |
| Polyvinylpyrrolidone (Povidone K90) | 4.00 | 79.14 | 27.98 | | 79.80 |
| Polysorbate 80 (Tween 80) | 0.10 | 2.02 | 0.71 | | 2.03 |
| Ethanol | 21.89 | | 155.60 | | |
| Total | 26.94 | 100.01 | 190.67 | | 99.99 |
| Area density [g/m²] | 55.4 | | 50.0 | | |
| Agomelatine content [µg/cm^{z}] | 551.6 | | 499.2 | | |
| Size of the OTF [cm²] | 0.522 | | | | |
| Backing layer | | | | | |
| | Ex. 3a | Ex. 3b | Ex. 3c | | Ex. 3d |
| Ingredient | | | Amount [g] | Solid [%] | FO-PET 15 µm |
| Ethylcellulose N50F | | | 5.22 | 65.09 | |
| Castor oil | | | 2.80 | 34.91 | |
| Ethanol | | | 45.33 | | |
| Total | | | 53.35 | 100.00 | |
| Area density [g/m²] | | | 12.3 | | |
| Size of the OTF [cm^{z}] | 0,522 | | | | |

Table 7

Agomelatine-containing matrix layer

| Ingredient | Ex. 3e - 3h | | | | | | | |
|---|---|---|---|---|---|---|---|---|
| | Amount [g] | | | | Solid [%] | | | |
| Agomelatine | 3.50 | | | | 9.98 | | | |
| Eucalyptol | 0.17 | | | | 0.49 | | | |
| Menthol | 0.35 | | | | 1.00 | | | |
| Methyl salicylate | 0.18 | | | | 0.51 | | | |
| Novamint Fresh Peppermint | 1.24 | | | | 3.52 | | | |
| Kolliphor RH 40 (Cremophor) | 0.72 | | | | 2.06 | | | |
| FD&C Red #40 | 0.04 | | | | 0.10 | | | |
| Sucralose | 0.18 | | | | 0.50 | | | |
| Polyvinylpyrrolidone (Povidone K90) | 27.98 | | | | 79.80 | | | |
| Polysorbate 80 (Tween 80) | 0.71 | | | | 2.03 | | | |
| Ethanol | 155.60 | | | | | | | |
| Total | 190.67 | | | | 99.99 | | | |
| Area density [g/m²] | 50.0 | | | | | | | |
| Agomelatine content [µg/cm²] | 499.2 | | | | | | | |
| Size of the OTF [cm²] | 0,522 | | | | | | | |
| Backing layer | | | | | | | | |
| | Ex. 3e | | Ex. 3f | | Ex. 3g | | Ex. 3h | |
| Ingredient | Amount [g] | Solid [%] | Amount [g] | Solid [%] | Amount [g] | Solid [%] | Amount [g] | Solid [%] |
| Kollidon VA 64 | 9.29 | 46.36 | 9.31 | 39.63 | 8.40 | 42.11 | 8.50 | 42.50 |
| Eudragit L100-55 | 9.22 | 46.01 | 9.21 | 39.21 | 8.40 | 42.11 | 8.50 | 42.50 |
| Glycerol (99.5%) | 1.54 | 7.63 | 1.51 | 6.39 | 1.55 | 7.72 | 3.02 | 15.00 |
| NaOH 0.1N | | | 3.47 | 14.77 | 1.61 | 8.07 | | |
| Ethanol | 22.49 | | 22.53 | | 22.5 | | 22.49 | |
| Purified water | 7.50 | | 7.55 | | 7.52 | | 7.56 | |
| Total | 50.04 | 100.00 | 53.58 | 100.00 | 49.98 | 100.01 | 50.07 | 100.00 |
| Area density [g/m²] | 26.8 | | 26.0 | | 20.5 | | 22.9 | |
| pH | 3.75 | | 5.01 | | 4.46 | | 3.62 | |
| Size of the OTF [cm²] | 0.522 | | | | | | | |

For Example 3a, a glass beaker was loaded with agomelatine. Ethanol, eucalyptol, menthol, methyl salicylate, Novamint Fresh Peppermint, Kolliphor RH 40, FD&C Red #40, sucralose and polysorbate 80 were added and the mixture was then stirred. The polyvinylpyrrolidone was added with stirring, and after about 2.5 hours of stirring a clear red solution was obtained.

For Examples 3b to 3h, the same coating composition was used for the agomelatine-containing layer, which was prepared as follows: A glass beaker was filled with agomelatine. Ethanol, menthol, eucalyptol, methyl salicylate, Kolliphor RH 40, FD&C Red #40, sucralose and polysorbate 80 were added and the mixture was then stirred. A clear solution was obtained. The polyvinylpyrrolidone was added and, after further stirring, the Novamint Fresh Peppermint was added dropwise with stirring to obtain a clear, red solution.

### Preparation of a Second Coating Composition (Backing Layer)

For Example 3c, a glass beaker was loaded with ethyl cellulose. Ethanol was added and the mixture was then stirred. Castor oil was added while stirring to obtain a slightly opaque mixture.

For Examples 3e to 3h, a glass beaker was loaded with ethanol. Purified water and Kollidon were added and the mixture was then stirred to obtain a solution. Eudragit and glycerol were added while stirring and a clear mixture was obtained.

For Examples 3f and 3g, sodium hydroxide solution was added to obtain a pH value as given in Table 7.

The resulting second coating composition of Examples 3c and 3e to 3h was applied to a polyester film (polyethylene terephthalate film, siliconised on one side, 75 µm thick, which can act as a separation layer) and dried for about 10 min at room temperature and 20 min at 70° C. (Ex. 3c) and for about 5 min at room temperature, 10 min at 35° C. and 2 min at 80° C. (Ex. 3e to 3h). The film thickness resulted in an area density of 12.3 g/m² (Ex. 3c), 26.8 g/m² (Ex. 3e), 26.0 g/m² (Ex. 3f), 20.5 g/m² (Ex. 3g) and 22.9 g/m² (Ex. 3h), respectively.

For Example 3d, a commercially available polyethylene terephthalate film with a thickness of 15 µm was used as the carrier layer.

The resultant agomelatine-containing first coating composition of Examples 3a and 3b was applied to a polyester film (polyethylene terephthalate film, siliconised on one side, 75 µm thick, which can act as a separation layer) and dried for about 15 min at room temperature and 5 min at 70° C. (Example 3a) or for about 5 min at room temperature, 10 min at 50° C. and 2 min at 90° C. (Example 3b). For Examples 3a and 3b, the dried film is the final agomelatine-containing layered structure.

The resultant agomelatine-containing initial coating compositions of Examples 3c and 3e to 3h were applied to the dried carrier layer and dried for about 5 min at room temperature, 10 min at 50° C. and 2 min at 90° C. (Ex. 3c and 3e to 3h).

The coating thickness resulted in an area density of 55.4 g/m² (Ex. 3a) or 50.0 g/m² (3b, 3c and 3e to 3h). The coating process of 3d was identical to that of Examples 3b, 3c and 3e to 3h, except that the coating composition was applied to a polyethylene terephthalate film of 15 µm thickness, resulting in an OTF with a carrier layer (of polyethylene terephthalate film).

The OTFs of Examples 3b, 3c and 3e to 3h were prepared by laminating the carrier layers to the agomelatine-containing layers. The separation layer was removed before lamination.

### Measurement of the Mucosal Permeation Rate

The permeate amount and the corresponding mucosal permeation rates of the OTFs prepared according to Examples 3a to 3h were determined by in vitro experiments according to the OECD guideline (adopted on 13 Apr. 2004) with porcine mucosa (mucosal oesophagus). A dermatome was used to produce mucous membrane up to a thickness of 400 µm with an intact barrier function. The OTFs were applied to the mucous membrane with an area of 0.522 cm² and the mucous membrane with the OTF on the upper side was immersed in artificial saliva (the underside is in contact with the receptor medium, the upper side is divided into a mucosal area of 1.145 cm²). The agomelatine permeated amount in the receptor medium (phosphate buffer solution pH 7.4) at a temperature of 37 ± 1° C. was measured and the corresponding mucosal permeation rate calculated.

The results are shown in Tables 8 and 9 and in FIGS. 3a and 3b. The standard deviation (SD) was calculated in this example using the n-method.

Table 8

| Time [h] | Ex. 3a (n = 3) | | Ex. 3b (n = 3) | | Ex. 3c (n = 3) | | Ex. 3d (n = 3) | |
|---|---|---|---|---|---|---|---|---|
| | Rate | SD | Rate | SD | Rate | SD | Rate | SD |
| 0.5 | 23.19 | 5.57 | 55.30 | 46.92 | 17.07 | 6.03 | 5.44 | 3.18 |
| 1 | 91.36 | 7.68 | 131.57 | 84.76 | 63.94 | 8.91 | 29.20 | 12.09 |
| 2 | 105.02 | 3.93 | 115.97 | 51.31 | 75.98 | 11.34 | 45.16 | 9.80 |
| 4 | 64.60 | 0.46 | 45.71 | 15.35 | 59.38 | 0.99 | 37.46 | 2.22 |
| 6 | 34.53 | 1.21 | 32.42 | 6.60 | 37.75 | 1.01 | 35.10 | 3.86 |

Table 9

| Time [h] | Ex. 3e (n = 3) | | Ex. 3f (n = 3) | | Ex. 3g (n = 3) | | Ex. 3h (n = 3) | |
|---|---|---|---|---|---|---|---|---|
| | Rate | SD | Rate | SD | Rate | SD | Rate | SD |
| 0.5 | 24.26 | 1.05 | 12.66 | 7.08 | 11.06 | 3.42 | 17.28 | 7.68 |
| 1 | 61.51 | 2.48 | 41.72 | 12.24 | 35.91 | 4.34 | 47.51 | 11.58 |
| 2 | 63.17 | 1.45 | 50.58 | 6.03 | 44.57 | 3.58 | 53.13 | 7.70 |
| 4 | 58.15 | 2.65 | 52.93 | 3.42 | 46.19 | 2.17 | 50.98 | 4.52 |
| 6 | 43.26 | 3.14 | 45.01 | 0.75 | 37.46 | 2.54 | 45.56 | 2.35 |

### Use of Agomelatine

The use of agomelatine after 6 hours was calculated based on the cumulative permeate amount after 6 hours and based on the initial agomelatine content. The results are shown in Table 10.

Table 10

Use of aqomelatine after 6 hours [%]

| Ex. 3a (n = 3) | Ex. 3b (n = 3) | Ex. 3c (n = 3) | Ex. 3d (n = 3) | Ex. 3e (n = 3) | Ex. 3f (n = 3) | Ex. 3g (n = 3) | Ex. 3h (n = 3) |
|---|---|---|---|---|---|---|---|
| 65.4 | 68.9 | 62.3 | 41.6 | 61.9 | 54.8 | 47.2 | 55.8 |

The in vitro experiments show a good mucosal permeation rate and a good use of the active agent. Examples 3c to 3h compared to Examples 3a and 3b show that even when a backing layer is used, a lower but still good permeation can be achieved.

1. A multi-layer oral thin film comprising a matrix layer, which contains at least one polymer and at least one pharmaceutically active agent, and at least one backing layer, wherein the at least one backing layer comprises at least one polymer containing free carboxyl groups, wherein 10 to 100% of the free carboxyl groups of the at least one polymer comprising free carboxyl groups are present in a neutralised form as a salt.

2. The multi-layer oral thin film according to claim 1, wherein the at least one backing layer has a pH of 3.5 to 7.

3. The multi-layer oral thin film according to claim 1, wherein the at least one polymer comprising free carboxyl groups is provided in the backing layer in an amount of 10 to 99 wt.%, in relation to the total weight of the backing layer.

4. The multi-layer oral thin film according to claim 1, wherein the content of free carboxyl groups in the polymer comprising free carboxyl groups is 10 to 40 wt.%, in relation to the mean polymer mass.

5. The multi-layer oral thin film according to claim 1, wherein the at least one polymer comprising free carboxyl groups comprises a polymer based on (meth)acrylic acid and/or based on a copolymer of (meth)acrylic acid and (meth)acrylates.

6. The multi-layer oral thin film according to claim 1, wherein the at least one polymer comprising free carboxyl groups comprises a (meth)acrylic acid/ethyl acrylate copolymer.

7. The multi-layer oral thin film according to claim 1, wherein the free carboxyl groups of the at least one polymer comprising free carboxyl groups have been neutralised by addition of at least one base.

8. The multi-layer oral thin film according to claim 1, wherein the backing layer comprises at least one plasticiser.

9. The multi-layer oral thin film according to claim 1, wherein the matrix layer comprises at least one water-soluble polymer.

10. The multi-layer oral thin film according to claim 9, wherein the at least one water-soluble polymer is selected from the group consisting of starch and starch derivatives, dextrans, cellulose derivatives, carboxymethyl cellulose, hydroxypropyl cellulose, hydroxyethyl cellulose, hydroxypropyl methylcellulose, hydroxypropyl ethyl cellulose, sodium carboxymethyl cellulose, ethyl or propyl cellulose, polyacrylic acids, polyacrylates, polyvinylpyrrolidones, vinyl pyrrolidone/vinyl acetate copolymer, polyvinyl alcohols, polyethylene oxide polymers, polyacrylamides, polyethylene glycols, gelatines, collagen, alginates, pectin, pullulan, tragacanth, chitosan, alginic acid, arabinogalactan, galactomannan, agar, agarose, carrageenan, and natural gums.

11. The multi-layer oral thin film according to claim 1, wherein the at least one pharmaceutically active agent is selected from the group consisting of the active agent classes of analgesics, hormones, hypnotics, sedatives, antiepileptics, analeptics, psychoneurotropic drugs, neuro-muscle blockers, antispasmodics, antihistamines, antiallergics, cardiotonics, antiarrhythmics, diuretics, hypotensives, vasopressors, antidepressants, antitussives, expectorants, thyroid hormones, sexual hormones, antidiabetics, antitumour active agents, antibiotics, chemotherapeutics and narcotics, wherein the at least one pharmaceutically active agent is preferably ketamine, especially preferably (S) ketamine.

12. The multi-layer oral thin film according to claim 1, wherein the matrix layer in each case also comprises at least one auxiliary substance selected from the group comprising colouring agents, flavourings, sweeteners, plasticisers, taste-masking agents, emulsifiers, enhancers, pH regulators, humectants, preservatives and/or antioxidants and the backing layer also comprises at least one auxiliary selected from the group comprising colouring agents, flavourings, sweeteners, taste-masking agents, emulsifiers, enhancers, pH regulators, humectants, preservatives and/or antioxidants.

13. A method for producing a multi-layer oral thin film according to claim 1, comprising the steps of:
a) providing at least one active agent-containing matrix layer, comprising the steps of
a1) producing a suspension or suspension comprising the at least one polymer and the at least one pharmaceutically active agent, and
a2) spreading out and drying the solution or suspension obtained in accordance with step a1);
b) providing at least one backing layer, comprising the steps of
b1) producing a solution or suspension comprising at least one polymer comprising free carboxyl groups, wherein 10 to 100% of the free carboxyl groups of the at least one polymer comprising free carboxyl groups are present in a neutralised form as a salt;
b2) spreading out and drying the solution obtained in accordance with step b1);
c) joining together the active agent-containing matrix layer obtained in accordance with a) and the backing layer obtained in accordance with b) in order to obtain a multi-layer oral thin film.

14. A multi-layer oral thin film obtained by the method according to claim 13.

15. A method for providing a pharmaceutically active agent comprising administering the multi-layer oral thin film according to claim 1.

16. The multi-layer oral thin film according to claim 1, wherein the free carboxyl groups of the at least one polymer comprising free carboxyl groups have been neutralised by addition of at least NaOH.

17. The multi-layer oral thin film according to claim 1, wherein the backing layer comprises at least triethyl citrate.
